(19) United States
(12) Patent Application Publication
(10) Pub. No.: US 2013/0129803 A1
(43) Pub. Date: May 23, 2013

(54) COSMETIC MATERIAL COMPRISING A SAPPAN WOOD EXTRACT ON A CLAY SUBSTRATE

(71) Applicants: Helene De Clermont-Gallerande, Vincennes (FR); Christine Dacruz, Choisy Le Roi (FR); Christine Brunet, La Jarne (FR); Anne De La Sayette-Saby, La Rochelle (FR)

(72) Inventors: Helene De Clermont-Gallerande, Vincennes (FR); Christine Dacruz, Choisy Le Roi (FR); Christine Brunet, La Jarne (FR); Anne De La Sayette-Saby, La Rochelle (FR)

(73) Assignee: CHANEL PARFUMS BEAUTE, Neuilly sur Seine (FR)

(21) Appl. No.: 13/813,475

(22) PCT Filed: Aug. 2, 2011
(86) PCT No.: PCT/EP11/63315

§ 371 Date: Jan. 31, 2013

(30) Foreign Application Priority Data
Aug. 5, 2010 (FR) 10/56459

(51) Int. Cl. A61K 8/02; A61Q 1/02; A61Q 1/06

(52) U.S. Cl. CPC A61K 8/0241; A61Q 1/06; A61Q 1/02
USPC 424/401; 424/63; 424/64

(57) ABSTRACT

A material including an organic dye of vegetable origin on an inorganic substrate, characterized in that the organic dye is an extract of sappan wood and in that the inorganic substrate is a clay which is a continuous sheet crystallized silicate. Also, a cosmetic composition containing this material, and the uses thereof.

The present invention concerns a material comprising an organic dye of vegetable origin fixed onto an inorganic substrate, characterized in that the said organic dye is a sappan wood extract and in that the said inorganic substrate is a clay which is a continuous sheet crystallized silicate. It also concerns a cosmetic composition containing this material and the uses thereof.

Cochineal carmine is a natural dye whose use dates back to the Aztecs and which, since it was introduced in Europe in the XVI^{th} century, has been widely used not only for the traditional uses thereof for painting and dyeing, but also in the food or cosmetic industry on account of its very characteristic purplish, vivid deep red colour.

Carmine is essentially produced in Peru, from female cochineal insects harvested on prickly pear cacti, which are dried in the sun and then boiled to produce a suspension containing the carminic acid released by the insects. The filtrate is then washed and dried before being fixed onto a substrate, usually calcium carbonate, to form a lake pigment intended to be used in industry.

This lake pigment has been the focus of renewed interest in recent decades as substitute for some synthetic dyes which it is sought to avoid. Synthetic red dyes are indeed prohibited in some products such as make-up, and many of these dyes are even prohibited in other products in some countries such as the United States. It nevertheless remains that, on account of its animal origin, cochineal carmine may give rise to supply problems, but it may also raise concerns at health level on account of allergy risks through ingestion in particular when used for lipsticks.

There therefore subsists a need to provide an alternative of vegetable origin to cochineal carmine.

The Applicant has discovered that an extract of sappan wood on a clay substrate which is a continuous sheet crystallized silicate allows this need to be met.

Sappan wood (or Caesalpinia sappan) is a thorny bush traditionally used in South-East Asia and in Indonesia as dye for producing paints, dyes or inks. It is also known to have anti-bacterial and anti-coagulant properties.

In application WO 01/55263, it was suggested to use brazilin contained in an extract of wood sappan (together with numerous other dye substances) to form a vegetable lake pigment to coat a dyed inorganic pigment which may itself be chosen from among compounds containing iron, chromium or manganese, in particular a clay coloured with iron oxides (such as sienna clay). It is indicated in this document that the lake layer allows the avoiding of direct contact between the skin and the inorganic core which may generate oxidative stress which would accelerate ageing of the skin. It is not envisaged to use this material as alternative to cochineal carmine.

In addition, the inventors have shown that the choice of substrate for sappan wood is critical in order to obtain a pigment having dye properties close to those of cochineal carmine in particular when it is mixed with white pigments, and that clays are a suitable substrate for this purpose.

The subject of the present invention is therefore a material comprising an organic dye of vegetable origin on an inorganic substrate, characterized in that the said organic dye is an extract of sappan wood and in that the said inorganic substrate is a clay which is a continuous sheet crystallized silicate.

Preferably, the clay used in the material according to the invention is not substantially coloured i.e. it contains less than 5% by weight of iron oxides, manganese and chromium after drying. Among the clay minerals a distinction is particularly made between continuous sheet crystallized silicates, fibrous clays and amorphous minerals (such as glass). The clay of the invention is a continuous sheet crystallized silicate.

More particularly, the clay of the invention can in particular be of 1:1 dioctahedral type preferably a kaolinite, or of 2:1 dioctahedral type preferably a smectite.

According to one preferred embodiment, the clay of the invention is a kaolinite.

Kaolinites, of formula Al₂Si₂O₅(OH)₄, are clay minerals of 1:1 type (which means that a sheet of kaolinite is formed of two layers: a tetrahedral layer SiO₄/an octahedral layer Al(OH—)₅O) characterized by low cation exchange with the exception of Fe³⁺ (x=0). The group of kaolinites includes dickite and nacrite which are particular cases of kaolinites characterized by different stacking of the same layers.

Preference is given in this invention to the use of clay of 1:1 di-octahedral type.

More preferably, the clay used as substrate is kaolin, which is a clay formed by decomposition of feldspars containing between 80 and 95% kaolinite and identified under the INCI name as “kaolin”. Kaolin may also contain other clays such as dickite, nacrite and halloysite (formed by decomposition of kaolinite subsequent to layering of water between the sheets).

Smectites are clays characterized by the presence of interlayer water and low cationic charge (x=0.2-0.6). They may comprise different exchangeable cations. A distinction is made between the dioctahedral smectites which include montmorillonite, beidellite and nontronite, trioctahedral smectites which include saponite, hectorite, stevensite, sauconite and talc. Preference in this invention is given to the use of a trioctahedral smectite which, in addition to silicon and magnesium, contains sodium such as stevensite, hectorite or saponite. Further preferably, the clay used as substrate is ghassoul or “rhassoul”, which is clay with natural saponins found in Morocco containing at least 90% stevensite and identified under the INCI name as “MOROCCAN LAVA CLAY”.

In addition to the aforementioned clay, the material of the invention contains an extract of sappan wood, which is preferably a hydro-alcoholic extract. Said extract can be obtained by crushing sappan wood followed by extraction in a hydro-alcoholic solvent formed of a mixture of water and a mono-alcohol such as ethanol or isopropanol, in a weight ratio of water to alcohol ranging from 20% to 80%, limits included. The weight ratio of sappan wood to solvent may also be between 1:10 and 1:25 limits included. Extraction can be conducted for a time of 2 h to 10 h, at a temperature ranging from 40 to 80° C. This gives a vegetable residue and a dye extract which is then filtered to remove the fine vegetable residues contained therein. This extract can be used as such to produce the material of the invention, or it can be transformed to a dry extract by evaporation of the solvent and drying—in particular oven or spray dying—if it is desired to defer the production of the material according to the invention. In this case it will need to be solubilised a further time in a hydro-alcoholic solvent in order to produce this material.

As a variant, the extract of sappan wood used according to the invention can be commercially obtained.

The material of the invention can in particular be obtained using a method comprising the following steps:

a) dispersing the clay in an aqueous phase,

b) adding alum to the said aqueous phase, at acid pH, to form an activated substrate dispersed in an aqueous medium,

c) adding to the said aqueous medium an extract of sappan wood previously solubilised in a hydro-alcoholic solvent,

d) adjusting the pH to a value of 5 to 9, to obtain a dispersion of material on a substrate,

e) filtering the said dispersion,

f) drying the material fixed onto the substrate.

This lake production method is conventional which means that persons skilled in the art will easily be able to adjust the parameters thereof, in particular the concentrations of the reagents and the pH values, so as to obtain a material having the desired colour, this colour possibly ranging from pink to purple passing through red. For example, to obtain a material having a colour close to cochineal carmine, the pH of the aqueous phase at step b) is preferably adjusted to a value of between 3 and 4, limits included, and at step (d) it is returned to a value of between 5 and 9, preferably between 6 and 8, limits included. Adjustment of the pH can be performed using a base such as sodium hydroxide or optionally an acid such as citric acid. In addition, in this form of embodiment, it is preferred that the weight ratio of alum to clay should be between 20:1 and 2:1, limits included, preferably between 5:1 and 12:1. It is also preferred that the weight ratio of sappan wood extract (dry matter) to clay should be between 1:10 and 1:1, limits included. The weight ratio of the sappan extract (dry matter) to the total weight of clay and alum may also be between 1:5 and 1:30, preferably between 1:10 and 1:24, limits included.

The method of the invention is also generally implemented at a temperature of between 20° C. and 80° C., preferably between 40 and 60° C., limits included, the drying step possibly being conducted at a temperature of between 30° C. and 60° C., limits included.

Evidently, this method may additionally comprise other preliminary, intermediate or subsequent steps to the above steps, provided that they are not detrimental to obtaining a material having the desired colour.

Having regard to its particular colour, the material of the invention can be used as pigment for colouring the lips and/or skin. In addition, the known anti-elastase, anti-oxidant and anti-collagenase properties of sappan wood extract allow the envisaging of the use of this material to prevent or attenuate signs of ageing of the lips (in particular to maintain the fleshiness of lips) and/or of the skin.

A further subject of the invention therefore concerns these uses.

For these applications, the above-described material is generally included in a cosmetic composition.

A further subject-matter of the invention is therefore a cosmetic composition containing this material, for example to the proportion of 0.1 to 60% by weight, relative to the total weight of the composition.

This composition usually contains a cosmetically acceptable medium i.e. able to be applied to the skin or lips without generating any discomfort (tightness, blotching, stinging) unacceptable for users.

The composition of the invention may be in the form of a lipstick or lip gloss. As a variant, it may be in the form of an eyeshadow and/or blusher. As a further variant, it may be in the form of a skincare product or foundation.

The composition of the invention may also be in any other galenic form in which these products are usually presented, in particular in the form of an anhydrous composition (i.e. containing less than 5% water by weight, even less than 1 weight %) or in the form of a water-in-oil or oil-in-water emulsion. This composition may take on the form of a powder, a paste, a cream, a gel or a fluid.

In addition to the aforementioned material, this composition may contain various conventional cosmetic additives as listed in particular in the CTFA Dictionary, 11^{th} Edition, 2006.

For example, it may in particular contain at least one oil.

In the meaning of the present invention, by <<oil>> is meant a compound liquid at ambient temperature (25° C.) and which, when added to the proportion of at least 1% by weight to water at 25° C., is not at all soluble in water, or soluble at up to less than 10 weight % relative to the weight of oil added to the water.

The oils which can be used in the composition of the invention may be chosen from among: hydrocarbonated oils; synthetic polyesters and polyethers and in particular polyesters of C₆-C₂₀ acids and C₆-C₂₀ alcohols, advantageously branched such as isononyl isononanoate; vegetable oils; branched and/or unsaturated fatty acids; branched and/or unsaturated fatty alcohols such as octyldodecanol; silicone oils such as straight-chain polydimethylsiloxanes, optionally phenylated, or cyclic: fluorosiliconated oils; fluorinated oils; and mixtures thereof.

Among these oils, for lipsticks or lip glosses, it is preferred to use at least one shiny oil i.e. an oil having a refractive index of more than 1.45 and preferably more than 1.47.

Examples of shiny oils are in particular hydrocarbonated oils such as polybutene, hydrogenated polyisobutene or hydrogenated polydecene, as well as phenylated silicone oils such as those identified under the INCI name <<phenyl trimethicone>>, of which one example is the silicone available under the trade name MIRASIL® PTM made by RHODIA, those identified under the INCI name <<phenylpropyldimethylsiloxysilicate>> of which one example is the silicone available under the trade name SILSHINE® 151 by GENERAL ELECTRIC, and those identified under the INCI name <<trimethyl pentaphenyl trisiloxane>> of which one example is the silicone available under the trade name DC PH® 1555 HRI produced by DOW CORNING.

As shiny oils the fluorinated silicones may also be mentioned, named under INCI as <<perfluorononyl dimethicone>> of which one example is the silicone available under the trade name PECOSIL® FS (FSU, FSL . . . ) by PHOENIX, and another example is the silicone available under the trade name Biosil Basics® (Fluorosil LF, 14 . . . ) produced by BIOSIL TECHNOLOGIES.

Other examples of shiny oils are natural oils and in particular castor oil; mono- and polyesters of fatty acids and/or of fatty alcohols whose fat chain contains 6 to 20 carbon atoms, and in particular: the mono- and polyesters of hydroxyacids and fatty alcohols such as diisostearyl malate, the esters of benzoic acid and of fatty alcohols such as the benzoate of C₁₂-C₁₅ alkyls, the polyesters of polyols and in particular of (di)pentaerythrityl such as pentaerythrityl tetraisostearate, dipentaerythrityl pentaisononanoate and the C₅-C₉ esters of dipentaerythrityl, or of polyglycerol such as the one known under the INCI name <<bis-diglyceryl polyacyladipate-1>> marketed by SASOL under the trade name SOFTISAN® 645, or of trimethylolpropane such as trimethylolpropane triethylhexanoate which is marketed in particular by KOKYU ALCOHOL KOGYO under the trade name KAK® TTO, or of propylene glycol such as dipropylene glycol dibenzoate marketed in particular by INOLEX under the trade name LEXFEEL SHINE®, and isocetyl stearoyl stearate; and the polyesters of hydrogenated castor oil such as the esters marketed by KOKYU ALCOHOL KOGYO under the trade names RISOCAST® DA-H and RISOCAST® DA-L.

Evidently, the composition of the invention may comprise mixtures of the aforementioned oils.

The fatty phase of this composition may also contain at least one wax. By <<wax>> is meant a fat having a melting point higher than 30° C. and generally lower than 100° C., which is liquid under the conditions for preparing the composition, and in the solid state it has an anisotropic crystalline organization. Examples of waxes are in particular animal, vegetable, mineral or synthetic waxes, the latter advantageously and possibly being hydrocarbonated or siliconized waxes. For example mention may be made of Chinese insect, Carnauba, Candelilla, rice, bees (Cera alba), polyethylene waxes, optionally functionalised, and paraffin wax as well as ozokerite, microcrystalline waxes, straight-chain C₁₄-C₂₂ fatty alcohols and the triesters of C₈-C₂₀ acids and of glycerine such as glycerine tribehenate, and mixtures thereof, this list not being limiting. Mention may further be made of acetylated glycol stearate marketed by VEVY under the trade name CETACENE®.

The fatty phase of this composition may also advantageously contain at least one lipophilic gelling agent.

Examples of lipophilic gelling agents are in particular the silicone polymers and more particularly the elastomers of organopolysiloxane. Amongst these, mention may be made of polymers at least partly cross-linked resulting from the reaction of an organopolysiloxane carrying unsaturated groups, such as vinyl or allyl groups located at the end in the centre of the chain, preferably on a silica atom, with another reactive siliconized compound such as an organohydrogeno-polysiloxane. These polymers are usually available in gel form in a volatile or non-volatile siliconized solvent or in a hydrocarbonated solvent. Examples of said elastomers are particularly those marketed by SHIN ETSU under the trade names KSG-6, KSG-16, KSG-31, KSG-32, KSG-41, KSG-42, KSG-43 and KSG-44, and by DOW CORNING under the trade names DC 9040 et DC 9041. Another oily gelling agent is formed of a silicone polymer obtained by self-polymerisation of an organopolysiloxane functionalised by epoxy groups and hydrosilylated in the presence of a catalyst, which is commercially available from GENERAL ELECTRIC under the trade name VELVESIL 125. Another lipophilic gelling agent is formed of a dimethicone/cyclic vinyldimethicone copolymer such as the one marketed by JEEN under the trade name JEESILC PS (including PS-VH, PS-VHLV, PS-CM, PS-CMLV and PS-DM). Another type of lipophilic gelling agent is formed of copolymers of styrene and olefins such as ethylene, propylene and/or butylene, optionally associated with siliconized or hydrocarbonated solvents such as those described in particular in application WO 98/38981 and in U.S. Pat. No. 6,309,629. They particularly comprise gelling agents containing sequenced terpolymers available from PENRECO under the trade name VERSAGEL. Another type of lipophilic gelling agent is composed of polyamides such as those identified under the INCI name polyamide-3 and in particular the polymers SYLVACLEAR AF 1900V and PA 1200V available from ARIZONA CHEMICAL and those identified by INCA as <<Ethylenediamine/Hydrogenated Dimer Dilinoleate Copolymer Bis-Di-C14-18 Alkyl Amide>> and available for example under the trade name SYLVACLEAR A200V or SYLVACLEAR A2614V from ARIZONA CHEMICAL. The lipophilic gelling agent, as a variant, may be a bentone.

The composition according to the invention may also contain at least one film-forming polymer in particular one able to provide wear resistance and/or non-transfer properties imparted by the composition to make-up.

This film-forming polymer may be lipophilic. It may in particular be a siliconized polymer, optionally urethane modified or fluorinated or acrylate such as the methacrylates marketed by SHIN-ETSU under the trade names KP-545, KP-561 et KP-562, or the polymers marketed by DOW CORNING under the trade names DC FA 4002 ID and DC FA 4001 CM. Other examples of film-forming polymers are silicon resins and in particular the MQ resins such as the trimethylsiloxysilicates and MT resins such as the derivatives of silsesquioxane and particularly the polymethylsilsesquioxanes marketed in particular by SHIN-ETSU, and polypropylsilsesquioxane marketed by DOW CORNING under the trade name DC 670, or phenylpropyl polysilsesquioxane marketed by WACKER under the trade name BELSIL SPR45VP. Another example is formed of fluoro-silicone polymers identified under the INCI name trifluoropropyldimethylsiloxy triethylsiloxysilicate such as the one marketed by GENERAL ELECTRIC under the trade name XS66-B8226. It is also possible as film-forming polymers to use bioadhesive polymers obtained, for example, by polycondensation of dimethiconol and MQ silicate resin in a solvent such as heptane, as marketed in particular by DOW CORNING under the trade names DC 7-4405 low tack and DC 7-4505 high tack. Other examples of film-forming polymers are the cyclic polyolefins such as polycyclopentadiene marketed in particular by KOBO under the trade name KOBOGUARD 5400, or polydicyclopentadiene. Further examples of film-forming agents are formed of copolymers of vinylpyrrolidone (VP) and/or straight-chain olefins such as the copolymers VP/hexadecene and VP/eicosene including ANTARON V216 and ANTARON V220 by ISP or the ethylene/vinyl acetate copolymers such as AC 400 by BAERLOCHER. Other film-forming polymers which can be used in this invention are polyacrylates such as poly(ethyl acrylate) marketed in particular by CREATIONS COULEURS under the trade name CREASIL 7 ID.

As a variant or in addition, the composition of the invention may contain at least one hydrophilic film-forming polymer and/or one or more hydrophilic gelling agents.

This composition may also contain one or more surfactants preferably chosen from among non-ionic surfactants, such as the esters of sorbitan optionally polyethoxylated, the esters of fatty acids and of glycerol, the esters of fatty acids and of sucrose, the esters of fatty acids and of polyethyleneglycol, polyether modified polysiloxanes, the ethers of fatty alcohols and of polyethyleneglycol, the alkylpolyglycosides and hydrogenated lecithin, this list not being limiting. It is preferred that the composition of the invention does not contain any anionic, cationic or amphoteric surfactant. By <<surfactant>> is meant a compound identified as such in the McCUTCHEON Dictionary.

The composition used according to the invention may also contain at least one filler. By this term is meant any particle of any shape (notably spherical or lamellar), mineral or organic, insoluble in the composition. Examples of fillers are talc, mica, silica, kaolin, boron nitride, starch, starch modified with octenylsuccinic anhydride, polyamides, silicone resins, silicone elastomer powders and acrylic polymer powders, in particular poly (methyl methacrylate). The fillers may in particular be formed of several layers of different chemical type and/or physical shape, and in particular they may be in the form of flakes coated with spherical fillers. They may also be modified using different surface treatments. One example of surface treated filler is formed of silica modified with an ethylene/methacrylate copolymer marketed in particular by KOBO under the trade names DSPCS 20N-I2, DSPCS/3H-I2 and DSPCS-I2.

The composition may also contain at least one other dye material in addition to the material of the invention, chosen from among water-soluble or liposoluble dyes, the effect of the fillers being to colour and/or opacify the composition and/or the skin such as colour pigments, nacreous pigments, lake pigments and mixtures thereof. These colouring materials may optionally be surface treated with a hydrophobic agent such as silanes, silicones, soaps of fatty acids, C₉-C₁₅ fluoroalcohol phosphates, acrylate/dimethicone copolymers, mixed C₉-C₁₅ fluoroalcohol phosphate/silicone copolymers, lecithins, carnauba wax, polyethylene, chitosan and amino acids optionally acylated such as lauroyl lysine, disodium stearoyl glutamate and aluminium acyl glutamate. The pigments may be mineral or organic, natural or synthetic. Examples of pigments are in particular iron oxides, titanium, chromium or zinc, ultramarine, Prussian blue, carbon black, as well as composite pigments and gonio-chromatic, pearlescent, interferential, photochromic or thermochromic pigments, this list not being limiting. Pearl pigments can be chosen from among those conventionally present in make-up products such as mica-titanium dioxides.

The composition of the invention may also contain anti-oxidant agents such as the alkylated or phosphorylated esters of ascorbic acid, or tocopherol and its esters; sequestering agents such as EDTA salts; pH adjusters; preserving agents; perfumes.

This composition may further contain at least one UV filter chosen from among organic and inorganic filters and mixtures thereof. As organic filters, particular mention may be made of derivatives of dibenzoylmethane (including butyl methoxydibenzoylmethane), the derivatives of cinnamic acid (including ethylhexyl methoxycinnamate), salicylates, para-aminobenzoic acids, β,β′-diphenylacrylates, benzophenones, the derivatives of benzylidene camphor, phenylbenzimidazoles, triazines, phenylbenzotriazoles and anthranilic derivatives. As inorganic filters, particular mention may be made of filters containing mineral oxides in the form of pigments or nanopigments, coated or uncoated and in particular containing titanium dioxide or zinc oxide.

The invention will be better understood in the light of the following examples that are non-limiting given solely for illustration.

### EXAMPLES

### Example 1

### Preparation of a Material According to the Invention

### a) Plant Extraction

After crushing, the sappan wood was macerated in a 50:50 water/ethyl alcohol mixture, at a plant/solvent ratio of 1:18. Extraction was conducted at a temperature of 60° C. for a time of 4 h.

The separation of the plant from the hydro-alcoholic solution of extract was performed by centrifugation. The extract solution thus harvested was concentrated in vacuo. The concentrate was then dried.

The dry extract was then reduced to powder by grinding.

### b) Preparation of the Plant Extract on a Substrate

The chosen clay substrate, Rhassoul, was placed in solution in the presence of an aluminium salt (double aluminium and potassium sulphate) at a weight ratio of 1:8.

The previously prepared Sappan extract was added to the above solution of Rhassoul+aluminium and potassium salt, such that the weight ratio of Sappan extract to the mixture of Rhassoul+aluminium salt was 1:18.

The Sappan was fixed on its substrate at a temperature of 50° C., and then the pH was adjusted using a solution of sodium hydroxide or citric acid at a pH of 6.0.

The Sappan extract on its substrate was then collected by filtering and oven dried before being ground.

### Example 2

### Comparative Colorimetric Analysis

The material obtained in Example 1 was ground to the proportion of 20% by weight in diisostearyl malate, until a fluid homogeneous paste was obtained. The same grinding was performed on the material prepared according to Example 1 but substituting the rhassoul substrate by a substrate of alumina, mica, silica or kaolin.

Each of these materials was added to a white paste of lip gloss in the presence of titanium dioxide and diisotearyl malate, to obtain a formulation having the following composition:
white paste of gloss: 89%
diisostearyl malate: 8.7%
extract of sappan on substrate: 2%
titanium dioxide: 0.3%
   and a spectrocolorimetric study was then conducted to compare the glosses with each other and with an identical gloss containing cochineal carmine.

The following parameters were precisely measured on a MINOLTA spectro-colorimeter CM-3600 D series (illuminant D65/10°; Lab colour space, SCI) coupled with Spectramagic NX software):
lightness L* (ranging from 0 for black to 100 for white)
red a*/green−a* (having greater presence the higher the absolute value)
yellow b*/blue−b* (having greater presence the higher the absolute value)
and the colour difference ΔE*:

ΔE*=\sqrt{(L₁−L₂)²+(a₁−a₂)²+(b₁−b₂)²}

The results of these measurements are grouped together in Table 1 below.

TABLE 1

| Tested | | | | |
|---|---|---|---|---|
| material | L* | a* | b* | ΔE* |
| Sappan + | 32.58 | 19.47 | 1.06 | 2.24 |
| rhassoul | | | | |
| Sappan + | 31.39 | 18.03 | −0.18 | |
| alumina | | | | |
| Sappan + | 36.02 | 24.71 | 0.89 | |
| mica | | | | |
| Sappan + | 40.79 | 29.89 | 1.94 | |
| silica | | | | |
| Sappan + | 33.67 | 24.58 | 1.30 | |
| kaolin | | | | |
| Cochineal | 28.53 | 20.16 | 7.22 | |
| carmine | | | | |

From this table it arises that the rhassoul-based and alumina-based materials used in a gloss, show a significant difference in colour ΔE* between them. More precisely, the comparative powder appears less red whilst the gloss obtained with the material of the invention has a colour closer to cochineal carmine i.e. a more vivid red.

The other tested materials appear much redder. However, the colour obtained is much too pale for the intended use.

### Example 3

### Incorporation of the Material in a Lip Gloss

A test was conducted conforming to Example 2 with the materials of Examples 1 and 2, except for the sappan wood fixed on a kaolin substrate for which another batch was used. The La*b* values obtained for the compound alone were compared with those of the compound when added to a white paste of lip gloss in the presence of titanium dioxide and diisotearyl malate as in Example 2.

| | | L* | a* | b* | ΔL* | Δa* | Δb* | ΔE*ab |
|---|---|---|---|---|---|---|---|---|
| Sappan | With | 23.88 | 2.29 | 0.3 | 8.70 | 17.18 | 0.76 | 19.27 |
| Rhassoul | TiO₂ | | | | | | | |
| | Without | 32.58 | 19.47 | 1.06 | | | | |
| | TiO₂ | | | | | | | |
| Sappan | With | 24.64 | 3.79 | 0.42 | 16.14 | 26.11 | 1.52 | 30.73 |
| Silica | TiO₂ | | | | | | | |
| | With | 40.79 | 29.89 | 1.94 | | | | |
| | TiO₂ | | | | | | | |
| Sappan | Without | 25.32 | 6.24 | 1.12 | 9.47 | 19.15 | 0.01 | 21.37 |
| | TiO₂ | | | | | | | |
| Kaolin | With | 34.8 | 25.39 | 1.13 | | | | |
| | TiO₂ | | | | | | | |

These tests show that the material containing sappan wood fixed on a kaolin substrate shows almost-zero variation in Δb* whereas it is 1.52 for example for sappan wood fixed on a silica substrate. Therefore, the sappan wood fixed on a kaolin substrate shows good preserving of its red colour even when associated with TiO₂.

### Example 4

### Incorporation of the Material in a Lip Gloss

The material obtained in Example 1 was ground in a triple roll grinder to the proportion of 20% by weight in diisostearyl malate. The grindings obtained were incorporated in a white paste pre-heated to 70-75° C., and the mixture was then cooled to 35° C. The gloss obtained had the following composition.

| INCI name/Function | weight % |
|---|---|
| Oils | 84.2 |
| Gelling agents | 10.5 |
| Material according to Example 1 | 4.0 |
| PHYTANTRIOL | 0.2 |
| PEG-8 | 0.1 |
| Preserving agents | 0.4 |
| Anti-oxidants | 0.6 |
| Total | 100.00 |

The physicochemical stability of this product was evaluated by placing in an oven for 15 days, 1 month and 2 months at different temperatures (5° C., 20° C., 40° C. and 45° C.), with evaluation also of UV resistance and make-up characteristics on the skin or lips.

UV resistance was evaluated by exposure to artificial light using Suntest® CPS+ equipment by ATLAS and an accelerated photo-ageing test in artificial light. The method is based on a comparative test between the product placed under the Suntest® and a control sample kept in the dark. The sun's radiation is simulated with an Arc Xenon light source whose light is filtered by 2 types of filters allowing both a reduction in the temperature of the samples and the cut-off of the shortest UV wavelengths. This Arc Xenon lamp gives off high heat, and to prevent thermal ageing from being stronger than photochemical ageing, a cooling system is used to cool the air of the Suntest® chamber throughout the exposure time of the samples. The products were observed and compared with the non-exposed control after different periods: 8 h, 24 h, 48 h. The discolouring observed was assessed to be acceptable or unacceptable.

No discolouring was observed compared with the control over the total duration of the test.

In addition, this product showed good lip coverage and was free of odour.

Finally, after accelerated ageing, it showed texture and rheology properties identical to those of the non-aged control, translating its good stability.

### Example 5

### Incorporation of the Material in an Eyeshadow

The material obtained in Example 1 was ground in a triple roll grinder to the proportion of 20% by weight in diisostearyl malate. The grindings obtained were incorporated in an eyeshadow having the following composition.

| INCI name/Function | weight % |
|---|---|
| Pigments and fillers | 84.2 |
| Fatty binders | 5.8 |
| Material according to Example 1 | 10.0 |
| Total | 100.00 |

For this purpose, the powder materials (pigments and fillers) were placed in a mixer where they were ground for twice 12 minutes. The fatty binder was then sprayed onto this powder mixture. The whole was then ground again for 3 times 10 minutes and screened before being compacted in a cup.

The physicochemical stability of this product was evaluated as described for Example 4.

Slight discolouring was observed on and after 24 h, however the product retained its original tone.

Also after applying to the eyelids, this product provided homogeneous make-up with good coverage and resistance without forming any particles.

Finally, it showed conforming brittleness and hardness values (i.e. identical to the non-aged control) thereby translating its good stability.

### Example 6

Incorporation of the material of Example 2 in a hot poured powder.

The material constituted of sappan wood fixed on a kaolin substrate obtained in Example 3 was ground in a triple roll grinder to the proportion of 10% by weight in isononyl isononanoate. The grindings obtained were incorporated in a hot flowed powder having the following composition.

| INCI name/Function | weight % |
|---|---|
| Pigments and fillers | 12 |
| Fatty binders (20-25% waxes having a melting | 78 |
| point ranging from 60 to 100° C. + 35% esters/ | |
| oils +/−20% elastomers/anhydrous gels | |
| Material according to Example 3 | 10.0 |
| Total | 100.00 |

For this purpose, the pigments and the isononyl isononanoate were placed in a ball miller where they were ground for 30 minutes.

The mixture of waxes and esters/oils was introduced in another miller and heated to 90° C. under stirring until the waxes are completely melted and the mixture becomes homogeneous.

The elastomers and anhydrous gels are then added to the miller under stirring at 80° C. until they become fully dissolved.

Finally, the mixture of pigments and isononyl isononanoate previously ground as well as the sappan wood fixed on a kaolin substrate obtained in Example 3 are further introduced in the miller. The mixture product is maintained at 80° C. for 15 minutes and then poured into a cup.

Color stability to temperature of the composition was then evaluated during the heating step of the preparation process of these hot poured powders.

For this purpose, a spectrocolorimetric measure was then conducted right after the addition of the mixture of pigments and isononyl isononanoate previously grounded as well as the sappan wood fixed on a kaolin substrate obtained in Example 3, by pouring the mixture in cups, and another spectrocolorimetric measure was conducted once the finished product is poured into cups at the end of the preparation process.

No discolouration of the product was observed during the heating step of the preparation process of the hot poured powders.

### Example 7

### Evaluation of the Anti-Age Effect

The sappan wood extract on its substrate of Example 1 was tested on in vitro cell cultures at concentrations varying from 0.00008% to 1.25 weight %, and compared with a non-treated positive control treated with EDTA.

It was found that the material in Example 1 significantly inhibited the activity of the metalloproteinases of MMP-1, MMP-2 and MMP-9 type, at least at some of the tested concentrations.

### Example 8

### Evaluation of the Anti-Oxidant and Anti-Age Effect

The sappan wood extract on kaolin substrate (batch used for Example 3) was tested on in vitro cell cultures at concentrations varying from 0.00008% to 1.25 weight % and compared with a non-treated control and a positive control treated with EDTA.

It was found firstly that the material of Example 3 containing kaolin significantly inhibited the activity of the 4 tested radicals (hydroxide radical OH°, superoxide anion O₂⁻, peroxynitrite ONOO— and halogenated oxidants), which demonstrates the anti-oxidant activity of the material of Example 3 containing kaolin.

It was also found that the material in Example 3 containing kaolin significantly inhibited the activity of type MMP-2 and MMP-9 metalloproteinases, at least at some of the tested concentrations.

1. Material comprising an organic dye of vegetable origin fixed on an inorganic substrate, characterized in that the said organic dye is an extract of sappan wood and in that the said inorganic substrate is a clay which is a continuous sheet crystallized silicate.

2. The material according to claim 1, characterized in that the said clay is a kaolinite, and preferably kaolin.

3. The material according to claim 1, characterized in that the said extract of sappan wood is a hydro-alcoholic extract.

4. The material according to claim 1, characterized in that it can be obtained using a method comprising the following steps:
a) dispersing the said clay in an aqueous phase,
b) adding alum to the said aqueous phase, at acid pH, to form an activated substrate dispersed in an aqueous medium,
c) adding, to the said aqueous medium, the said sappan wood extract previously solubilised in a hydro-alcoholic solvent,
d) adjusting the pH to a value ranging from 5 to 9, to obtain a dispersion of material on substrate,
e) filtering the said dispersion,
f) drying the material fixed onto the substrate

5. A cosmetic composition containing a material according to claim 1.

6. The composition according to claim 5, characterized in that it is in the form of a lipstick or lip gloss.

7. The composition according to claim 5, characterized in that it is in the form of an eyeshadow and/or blusher.

8. The composition according to claim 5, characterized in that it is in the form of a skincare product or a foundation.

9. A method of preventing or attenuating signs of ageing of the lips and/or of the skin, comprising applying material according to claim 1 to the lips and/or the skin of a subject in need thereof.

10. A method of colouring lips and/or skin, comprising applying the material according to claim 1 as a pigment to the lips and/or skin.
